(19) United States
(12) Patent Application Publication
(10) Pub. No.: US 2025/0359752 A1
(43) Pub. Date: Nov. 27, 2025

(54) DEVICE FOR MEASURING MAXWELL SPOT CENTROID ORIENTATIONS, PARTICULARLY IN PEOPLE WITH DYSLEXIA

(71) Applicant: Albert LE FLOCH, Rennes (FR)

(72) Inventor: Albert LE FLOCH, Rennes (FR)

(21) Appl. No.: 18/872,691

(22) PCT Filed: Jun. 5, 2023
(86) PCT No.: PCT/IB2023/055777

§ 371 Date: Dec. 6, 2024

(30) Foreign Application Priority Data
Jun. 8, 2022 (FR) 2205481

(51) Int. Cl. A61B 3/10; A61B 3/00; G02B 26/00

(52) U.S. Cl. CPC A61B 3/1005; A61B 3/0008; A61B 3/0033; G02B 26/008

(57) ABSTRACT

An electronic device is disclosed that is able to measure the critical orientations of the ellipses with a precision of 2°, and respective orientations of the main axes for both eyes, which lead to reading difficulties in children. The device according to the invention comprises a control unit for controlling the orientation of an elliptical profile in order to be able to measure mirror-image or translational orientations that are liable to lead to reading difficulties. Opposite or parallel orientations of two profiles having the same ellipticity may in particular lead to two types of dyslexia. The entopic image of each Maxwell spot centroid along with its orientation, observed using a viewing system with a double blue-green rotary filter, makes it possible to diagnose various types of dyslexia. Advantageously, the rotary system makes it possible to choose the rotational frequency so as to optimize contrast quality.

### 1. FIELD OF THE INVENTION

The present invention concerns a device for measuring all cases of orientation among Maxwell spot centroid profiles, including elliptical profiles, for both foveas, in particular for a person with reading difficulties, and this for all possible orientations of the major axes of the ellipses.

### 2. STATE OF THE ART

Dyslexia is usually defined as a complex learning deficit, particularly in reading, which appears in childhood. Despite the different approaches that have been proposed, from biology, neurology, cognitive science and genetics, all converge on a biological origin linked to neurology and brain connectivity. This deficiency is widespread, affecting around 10% of a given population according to international statistics, with all countries equally affected.

Vision, with both eyes providing the two most important masses of information for the brain, but slightly different from the visual cortex, has been suspected of being involved since the early 20^{th} century. The visual clutter and confusion of symmetrical letters like “b” and “d”, and even of words and their mirrors, led Ernest Mach (The analysis of sensations, Chicago: Open Court 1914) to write: “To remove the confusion of the b-d type, an asymmetry must be introduced somewhere in the brain.” Furthermore, recent results from functional magnetic resonance imaging have shown that dyslexics have too little lateralization of the brain to perform certain functions. Reading, in particular, uses the left cerebral hemisphere for around 98% of the population. An asymmetry was detected between Maxwell spot centroids, that is, between the two small regions devoid of blue cones (of the order of 100 microns in diameter) in the center of the two foveas, for each person with no reading deficit (typical readers). A patent (Le Floch, Bourdillon, Ropars) was filed on Nov. 6, 2012 with the INPI (FR2997618) for a device to detect this asymmetry in typical readers. For people with ocular instabilities, an initial lack of asymmetry was found, but it was not until 2017 that the first dyslexic people were found with circular Maxwell spot centroids, without asymmetries, with excess mirror or duplicate images that cause visual clutter making reading difficult (Le Floch A., Ropars G., 2017, Proc. Soc. B, https://doi.org/10.1098/rspb.2017.1380). However, the lack of asymmetry in people with dyslexia is not limited to the case of circular contours. In fact, a lack of asymmetry may be observed between the two elliptical profiles for a dyslexic person. However, an error was made in this manuscript on page 5, where it states “and there is no preferred azimuth φ (FIG. 1d) for the elliptical outline.” Contrary to what was written in the 2017 article, it is not enough to have two circular contours. Indeed, a dyslexic may have two elliptical contours, but then the fundamental parameter is the orientation, which needs to be measured precisely. The purpose of this invention is to measure this orientation of the major axes for each eye, which should also enable early diagnosis for children. Indeed, parents suspect difficulties as early as 4-5 years of age, difficulties often highlighted by teachers as early as kindergarten but only confirmed by various reading tests, particularly around 8-9 years of age, carried out by speech therapists. The aim here, then, is not to propose a device for measuring the asymmetry that establishes ocular dominance in people without problems, but on the contrary to propose a device for comparing orientations, including in the presence of ellipticities in people with dyslexia.

### 3. BRIEF DESCRIPTION OF THE FIGURES

The invention will be better understood and other features and advantages will become apparent from the following description, which refers to the attached figures, among which:

FIG. 1 is a schematic diagram of the architecture of the device comprising a control and measurement unit UCM for the fundamental parameters of the ellipse, essentially the orientation, projected by a projector P onto a brightly lit screen E and the viewing system V of the observer O with a dual rotary filter FB (blue filter) and FV (green filter) according to a non-limiting embodiment of the invention.

FIG. 2 defines the relevant parameter to be measured, namely the angle φ giving the orientation of the two main vertices A and B with respect to the vertical. C and D are the secondary vertices of the ellipse, which also allow us to measure the ellipticity defined by ϵ=CD/AB. If we locate the orientation of the major axis φ_{D} for the right eye and φ_{G} for the left eye, this is the difference φ_{D}−φ_{G} that is crucial.

FIG. 3 measures the case of persons with indeterminate orientations and differential ellipticity (Δϵ=0).

FIG. 4 measures the case, also with zero differential ellipticity, but with equal “mirrored” orientations, i.e., with opposite orientations (example: φ_{G}=−40°, φ_{D}=+40°).

FIG. 5 shows the case with identical orientations and zero differential ellipticity, but with equal “translational” orientations, i.e., parallel orientations (e.g., φ_{G}=+40°, φ_{D}=+40°).

### 4. DETAILED DESCRIPTION OF THE INVENTION

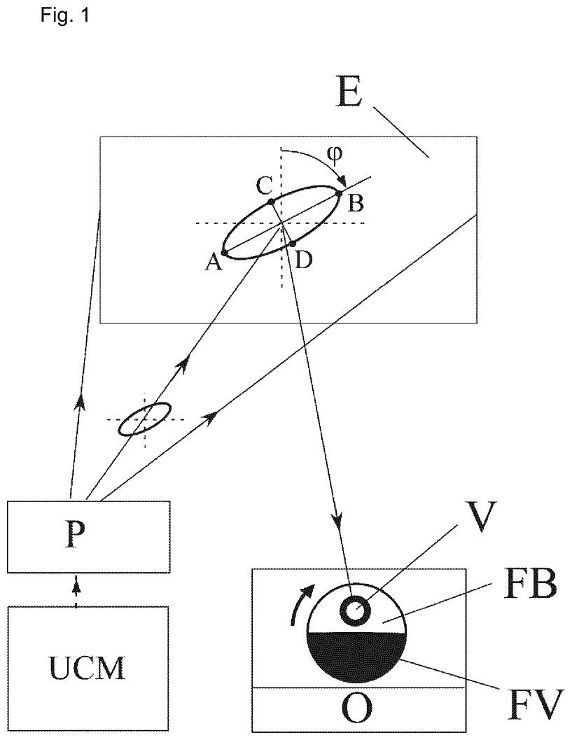

In FIG. 1, the modules shown are functional units which may or may not correspond to physically distinguishable units. For example, some or all of these modules are grouped together in a single component. On the other hand, in other embodiments, some modules are made up of physically separate entities. E denotes a screen illuminated with white light; P denotes a projector; UCM denotes a unit for controlling and measuring the orientation, ellipticity and elliptical surface of Maxwell spot centroids; V denotes the viewing system of observer O; FB and FV denote a blue filter and a green filter, respectively.

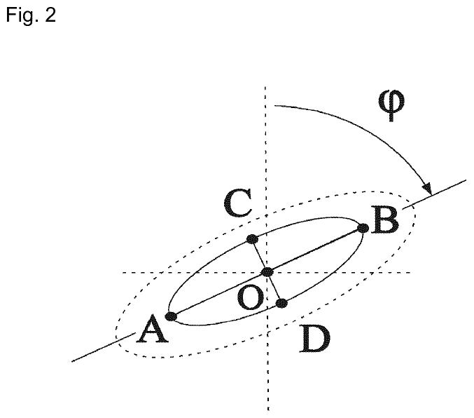

FIG. 2 introduces all the parameters used in the invention: the main vertices A and B establishing the major axis and defining the orientation φ for each eye of the elliptical contours, crucial in this case. The display on the device then shows all orientations φ_{D} and φ_{G} the respective orientations of the two major axes for the right and left eyes, and the difference.

Advantageously, the parameter controller diagrammed in FIG. 1 can be used to adjust the orientation of the major axis to within 2° for each eye in turn, via an electronic card. The projector is set to evenly illuminate the screen in white light, with a luminous flux of between 2000 and 4000 lumens.

Advantageously, the light beam is produced from one or more elements of the LED type, from the acronym “Light Emitting Diode”. Of course, the light beam can be created using other lighting elements that provide stable illumination sufficient to visualize Maxwell spot centroids with good contrast, such as LEDs of different colors, like blue and green.

Advantageously, the sighting system featuring a rotating blue-green dual filter uses a small electric motor to vary the rotation frequency between 0.1 Hz and 1 Hz, at the observer's convenience. The sighting system-screen distance of around 3 m creates a Maxwell spot centroid at screen level, with an average diameter of around 3 cm, enabling suitable superposition with the projected elliptical profiles.

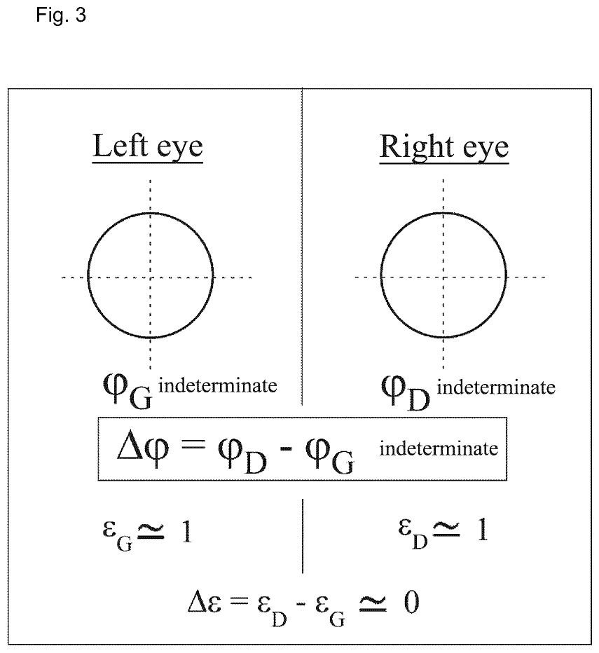

In the case of FIG. 3, the device allows the observation and recording of parameters for the most common situation, where in particular the dyslexic person sees two contours of quasi-circular Maxwell spot centroids where φ_{D}, φ_{G} and the difference Δφ=φ_{D}−φ_{G} are indeterminate and where ϵ_{D}≃ϵ_{G}≃1.

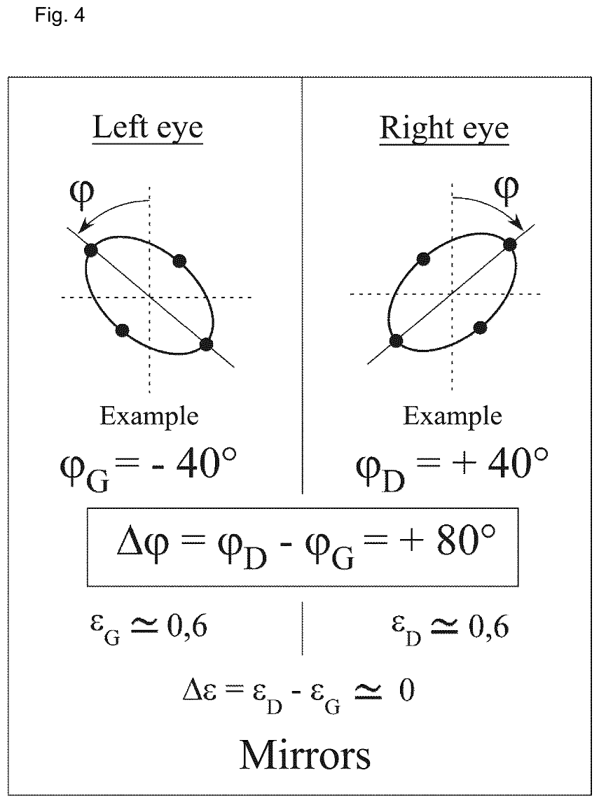

Advantageously, the device makes it possible to identify the distribution shown in FIG. 4, which certainly corresponds to zero differential ellipticity (Δϵ≃0 with ϵ_{D}≃ϵ_{G}≠1), but orientations OD and OG opposite (mirrored) major axes with respect to a vertical axis (φ_{D}=−φ_{D}).

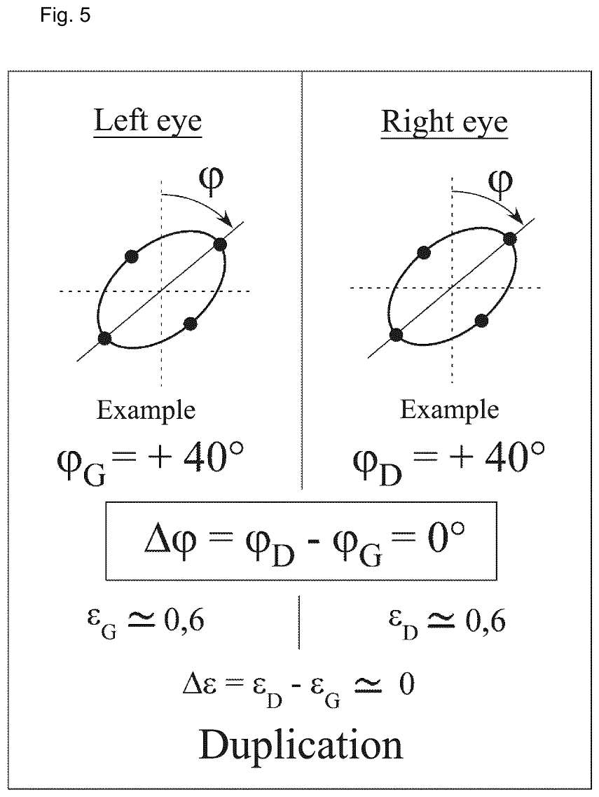

Advantageously, the device can also be used to qualify and measure situations of splitting by interhemispheric projections that are not translationally symmetrical, as shown in FIG. 5. Here again, the ellipticities are certainly equal (ϵ_{D}≃ϵ_{G} but ≠1) but the device of the invention can measure the orientations and parallelism of the main axes (φ_{D}=φ_{G}), for any value of the orientation of the two profiles.

The invention is not limited to the embodiments described above, but applies to any device for measuring Maxwell spot centroid orientations, including quasi-circular profiles, profiles with differential yet zero ellipticity, but with opposite or parallel orientations of the major axes, leading to reading deficits, particularly for people with dyslexia.

1. A device for measuring orientations related to possible ellipticities of Maxwell spot centroids devoid of blue cones of each eye in people with dyslexia, characterized in that it comprises:
an electronic control and measurement system UCM creating an ellipse with a major axis orientation adjustable to within 2°, projected onto a screen;
a lighting system of an order of 2,000 to 4,000 lumens, illuminating a screen on which the ellipse's contour is projected simultaneously, with tracking and display of the major axis orientations for both eyes and their angular difference.

2. The device according to claim 1, characterized in that it comprises means for measuring and displaying the orientations of the major axes of the two ellipses and their difference, between the two Maxwell spot centroids, so as to be able to define a type of dyslexia.

3. The device according to claim 1, in that it measures the difference between the orientations to within 2° of two ellipses of the same ellipticity for the left and right eyes, but with opposite orientations of the principal axes, leading to the first type of dyslexia.

4. The device according to claim 1, in that it enables measurement of profiles of the same ellipticity, but with parallel orientation of the main axes, leading to the second type of dyslexia corresponding to duplication of letters and words, particularly in certain people with dyslexia with reading difficulties due to duplication.

5. The device according to claim 1, including systems that may include colored LEDs for visualizing Maxwell spot centroids and characterizing the various asymmetry gaps mentioned above, particularly in people with reading difficulties.
